(19) United States
(12) Patent Application Publication
(10) Pub. No.: US 2020/0172687 A1
(43) Pub. Date: Jun. 4, 2020

(54) MEDICAL RADIATION ATTENUATION NATURAL RUBBER THIN FILMS, METHODS OF MAKING AND ARTICLES MADE THEREWITH

(71) Applicant: Ohio State Innovation Foundation, Columbus, OH (US)

(72) Inventors: Katrina Cornish, Columbus, OH (US); Zhenyu Li, Columbus, OH (US)

(73) Assignee: Ohio State Innovation Foundation, Columbus, OH (US)

(21) Appl. No.: 16/636,421

(22) PCT Filed: Aug. 2, 2018
(86) PCT No.: PCT/US2018/044909

§ 371 Date: Feb. 4, 2020

Related U.S. Application Data
(60) Provisional application No. 62/541,266, filed on Aug. 4, 2017.

(51) Int. Cl. C08J 5/18; C08K 3/22; C08K 3/30; C08K 5/39; C08K 5/38; A61B 42/10; A41D 19/00; A41D 19/04; G21F 1/08; G21F 3/035

(52) U.S. Cl. CPC C08J 5/18; C08K 3/22; C08K 3/30; C08K 5/39; C08K 5/38; A61B 6/107; A41D 19/0062; A41D 19/04; G21F 1/08; G21F 3/035; C08J 2307/02; A61B 42/10

(57) ABSTRACT

Medical radiation attenuation thin films, methods of making the same, and articles such as gloves made therefrom, are disclosed. The thin films utilize guayule natural rubber, sulfur and an attenuation filler such as Bi₂O₃. The films mix the guayule natural rubber, sulfur and attenuation filler and cure the mixture at about 80 to about 105° C. for about 40 to about 90 minutes.

### CROSS-REFERENCE TO RELATED APPLICATIONS

The present invention claims priority to U.S. provisional application Ser. No. 62/541,266, filed under 35 USC § 111(b) on Aug. 4, 2017, the disclosure of which is hereby incorporated by reference in its entirety.

### STATEMENT REGARDING FEDERALLY SPONSORED RESEARCH

The invention was made with no government support. The government has no rights in the invention.

### BACKGROUND OF THE INVENTION

There are at least 40,000 commercial products made from natural rubber (NR). Some examples of such products include clothing elastic, balloons, medical gloves, catheters, dental dams, and condoms.

Radiation attenuation (RA) gloves protect and shield health care professionals (HCP) from occupational exposure to ionizing radiation. Potential end-users include radiologists, cardiologists, surgeons, and technicians who administer radiation examinations and/or perform radiation treatments. For example, the types of procedures involved with radiation exposure to HCP include diagnostic arteriography, fluoroscopy assisted orthopedic procedures, and interventional cardiovascular procedures. In 2016 this number totaled over 16 million in the US alone.

In addition, several types of fluoroscopy-assisted surgeries require the hands of the HCP to be in or near the primary radiation field. Therefore, of all body parts, the hands usually receive the highest cumulative levels of radiation. Known outcome of overexposure is radiation dermatitis and skin damage. Stochastic effects of skin cancer cannot be ruled out. Any additional amount of radiation received is bound to pose additional occupational health hazard. Therefore, it is recommended for HCP to carry out sufficient protective measures to reduce cumulative exposure dosage. Wearing radiation attenuation (RA) protective gloves can reduce hand exposure by over 40%, thus lower the accumulative doses and associated long-term health risk.

Commercially available disposable RA gloves differ by base elastomer, thickness, tensile properties and degree of attenuation, with attenuation levels depending on the loading of the filler attenuation compounds and film thickness. Most RA gloves are formulated with hevea natural rubber (HNR) because of the higher filler capacity and tensile properties compared to synthetic rubber materials. However, the amount of radiation-attenuating diluent fillers still causes these gloves to fail the medical glove performance standards (See Table 1 below, ASTM D3577 for surgical gloves, and D3578 for examination gloves).

TABLE 1
Specification for Rubber Surgical/Examination Gloves (Natural)

| | | [symbol] imum | [symbol] inimum Tensile | [symbol] inimum Ultimate | [symbol] ximum Modulus |
|---|---|---|---|---|---|
| Standard # | Polymer Type | Thickness (mm) | Strength (MPa) | Elongation (%) | at 500% strain (MPa) |
| STM D3577 | [symbol] rgical I (natural) | 0.10 | 24 | 750 | 5.5 |
| STM D3578 | [symbol] xam I (natural) | 0.08 | 18 | 650 | 5.5 |

[symbol]

indicates data missing or illegible when filed

According to FDA regulations, this necessitates double gloving, in which a medical glove must also be worn, to protect against pathogen transmission. The RA gloves are already much thicker than normal medical gloves, and with double-gloving, tactile sensation and hand dexterity is further reduced to the potential detriment of surgical outcome.

Thus, there remains an unmet need in the RA protective garment industry for thin film barriers that provide sufficient radiation shielding and possess adequate mechanical properties for use in a wide variety of medical applications.

Such medical RA gloves should meet both the ASTM surgical glove standard (D3577) for tensile strength, ultimate elongation, and modulus, and the ASTM D7866 standard for radiation transmission attenuation factor of at least about 29% of a primary 60 kVp x-ray beam; at least about 22% of a primary 80 kVp x-ray beam; at least about 18% of a primary 100 kVp x-ray beam; and, at least about 15% of a primary 1000 kVp x-ray beam.

Such medical RA gloves should eliminate the FDA requirement for end-users to double glove with both an attenuation protective glove and a medical glove. Such improvement would be beneficial for the outcome of intraoperative fluoroscopy assisted surgical operations. Additionally, the medical RA gloves should avoid both contact and systemic latex allergies and skin irritation.

### SUMMARY OF THE INVENTION

In a first broad aspect, provided herein are films comprising guayule natural rubber, and one or more radiation attenuation fillers.

In certain embodiments, the film is formed into a medical radiation attenuation glove.

In one embodiment, the thin film is comprised of: guayule natural rubber, sulfur, and a radiation attenuation filler, where the film has a thickness of about 0.08 to about 0.40 mm, and a percent attenuation of at least about 29% at 60 kVp, at least about 22% at 80 kVp, at least about 18% at 100 kVp, and at least about 15% at 120 kVp; and, where the film is formed at curing temperatures of about 80 to about 105° C. for about 40 to about 90 minutes.

In certain embodiments, the sulfur is present at about 3.2 to about 3.6 per hundred rubber (phr).

In certain embodiments, the radiation attenuation filler is present at about 120 to about 200 phr.

In certain embodiments, the attenuation filler comprises one or more of: bismuth tri-oxide (Bi₂O₃), barium sulfate (BaSO₄), barium carbonate (BaCO₃), tungsten tri-oxide (WO₃), and tungsten (W).

In certain embodiments, he attenuation filler comprises Bi₂O₃ at about 120 to about 200 phr.

In certain embodiments, the film has a thickness of about 0.24 mm to about 0.31 mm.

In certain embodiments, the radiation attenuation examination has: a thickness of about 0.08 to about 0.40 mm, a tensile strength of at least about 18 MPa, an elongation at break of at least about 650%, and a 500% modulus of at most 5.5 MPa.

In certain embodiments, the radiation attenuation surgical glove has: a thickness of about 0.10 to about 0.40 mm, a tensile strength of at least about 24 MPa, an elongation at break of at least about 750%, and a 500% modulus of 5.5 at most MPa.

In certain embodiments, the film further includes accelerators comprising diisopropyl xanthogen polysulphide (DIXP) and zinc diisononyl dithiocarbamate (ZDNC).

In certain embodiments, the DIXP is present at about 2 PHR, and the ZDNC is present at about 0.8 phr.

In certain embodiments, the ZDNC can be present at a dry weight concentration ranging from about 0.01 phr to about 3 phr.

In certain embodiments, the DIXP is present at a dry weight concentration ranging from about 0.01 phr to about 5 phr.

In certain embodiments, DIXP and ZDNC are present in a ratio of DIXP:ZDNC of about 2.5:1 or less.

In certain embodiments, the film further includes one or more of: ammonium hydroxide,

ZnO, and one or more antioxidants.

In certain embodiments, the film comprises: about 100 phr rubber of guayule natural rubber; about 0.01 to about 5 phr of sulfur; about 120 to about 150 phr of at least one radiation attenuation filler; about 0.2 to about 1.4 phr of ZDNC; and about 1 to about 2.2 phr of DIXP.

In certain embodiments, the film comprises: about 100 phr of guayule natural rubber;

about 0.01 to about 5 phr of sulfur; about 120 to about 150 phr of at least one radiation attenuation filler; about 0.5 to about 1.4 phr of ZDNC; and about 1 to about 2.2 phr of DIXP.

In another aspect, there is provides a method for making a glove, that comprises:

combining guayule natural rubber latex, sulfur, and an attenuation filler, and forming the glove by dipping followed by curing at about 80 to about 105° C. for about 40 to about 90 minutes.

In yet another aspect, there is provided a method for making a glove, comprising the following steps: 1a) preheat former at about 70° C. for about 30 minutes, or 1b) start process at ambient temperature; 2) dip former into coagulant for about 1 to about 10 seconds; 3a) dry coagulant on former at about 70° C. for about 20 to about 30 minutes, or 3b) dry coagulant on former at ambient temperature; 4) dip former with dried coagulant into compound for about 30 to about 45 seconds; 5a) dry compound on the former at about 70° C. for about 25 to about 35 minutes, or 5b) dry compound on the former at ambient temperature; 6) optionally, perform hand beading; 7a) water leach at about 50 to about 70° C. for about 3 minutes, or 7b) water leach at ambient temperature for about 3 minutes; 8a) dry at about 70° C. for about 3 minutes, or 8b) dry at ambient temperature for about 3 minutes; 9) dip into polymer coating for about 3 to about 5 seconds; 10) vulcanize at about 80 to about 100° C. for about 40 to about 90 minutes; 11) cool down to ambient temperature; 12) remove glove from former, wash if necessary; 13) wash glove in detackifying lubricant solution; and, 14) tumble dry glove at low heat for about 60 minutes.

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIGS. 1A-1D: Color variation of fabricated GNR-Bi₂O₃ film samples. Photos taken from former side of the films dipped with thick formers and vulcanized at 90° C. for (FIG. 1A) 40 min, (FIG. 1B) 50 min, (FIG. 1C) 60 min, and (FIG. 1D) 70 min, respectively.

FIG. 2: Heating and cooling profiles vary between the thick and thin plate formers.

FIG. 3: Film thickness is significantly affected by former used, but less likely by added water. Treatments with different letter notations are significantly different (p<0.01). Error bar represents standard deviation of samples. Thick plate, 50 phr (parts per hundred rubber) water: mean of 0.2688±0.0126, n=16; Thick plate, 24 phr water: mean of 0.2905±0.0126, n=40; Thin plate, 24 phr water: mean of 0.2817±0.0017, n=12.

FIG. 4: Tensile stress variation from sulfur and bismuth tri-oxide loadings in phr. Treatments with different letter notations are significantly different (p<0.01). Error bar represents standard deviation of samples. 2.5 S, 150 Bi₂O₃: mean of 19.09±1.85, n=24; 3.2 S, 150 Bi₂O₃: mean of 23.47±2.38, n=12; 3.4 S, 150 Bi₂O₃: mean of 22.43±2.29, n=24; 3.4 S, 120 Bi₂O₃: mean of 26.54±1.968, n=24

FIG. 5: Tensile strain variation from sulfur and bismuth tri-oxide loadings in phr. Treatments with different letter notations are significantly different (p<0.01; a and a′, p<0.05). Error bar represents standard deviation of samples. 2.5 S, 150 Bi₂O₃: mean of 774.5±31.1, n=24; 3.2 S, 150 Bi₂O₃: mean of 805.2±24.0, n=12; 3.4 S, 150 Bi₂O₃: mean of 744.2±36.4, n=24; 3.4 S, 120 Bi₂O₃: mean of 759.1±28.5, n=24.

FIG. 6: Modulus at 500% strain from sulfur and bismuth tri-oxide loadings in phr. Treatments with different letter notations are significantly different (p<0.01). Error bar represents standard deviation of samples. 2.5 S, 150 Bi₂O₃: mean of 2.98±0.38, n=24; 3.2 S, 150 Bi₂O₃: mean of 2.83±0.21, n=12; 3.4 S, 150 Bi₂O₃: mean of 4.16±0.64, n=24; 3.4 S, 120 Bi₂O₃: mean of 4.34±0.51, n=24.

FIG. 7: Tensile performances by curing temperature and compounding formulation in phr. Based on data from all 84 samples. Curing temperature for each compounding formula is determined (** for both parameters passing surgical glove standard; * for only one). Dotted line represents minimum tensile strength and ultimate elongation requirement of surgical glove standard.

FIG. 8: Tensile performances by vulcanization condition, based on data from 20 samples made with 150 phr Bi₂O₃ and 3.4 phr sulfur loadings. Vulcanization time for each vulcanization temperature is determined (** if both parameters passing surgical glove standard; * for only one). Dotted line represents minimum tensile strength and ultimate elongation requirement of surgical glove standard.

FIG. 9: Photograph showing a film having increased water which caused Bi₂O₃ filler sediment.

FIG. 10: The appearance of example GNR-Bi₂O₃ RA medical gloves produced at lab scale: before vulcanization (left); and, after vulcanization (right).

FIG. 11: Example of a factory scale GNR-Bi₂O₃ medical RA glove fabrication process.

### DETAILED DESCRIPTION OF THE INVENTION

Various embodiments are described in the present disclosure in the context of latex compounds, thin films, methods of making thin films, and methods of using thin films. Those of ordinary skill in the art will realize that the following detailed description of the embodiments is illustrative only and not intended to be in any way limiting. Other embodiments will readily suggest themselves to such skilled persons having the benefit of this disclosure. Reference to an “embodiment,” “aspect,” or “example” in this disclosure indicates that the embodiments of the invention so described may include a particular feature, structure, or characteristic, but not every embodiment necessarily includes the particular feature, structure, or characteristic. Further, repeated use of the phrase “in one embodiment” does not necessarily refer to the same embodiment, although it may.

Not all of the routine features of the implementations or processes described herein are shown and described. It will, of course, be appreciated that in the development of any such actual implementation, numerous implementation-specific decisions will be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking for those of ordinary skill in the art having the benefit of this disclosure.

Definitions

For convenience, certain terms employed in the specification, examples, and appended claims are collected here, before further description of the invention. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used to refer to one or to more than one (i.e., to at least one) of the grammatical object of the article. By way of example, “an element” means one element or more than one element.

The term “plurality” means more than one.

The terms “comprise” and “comprising” are used in the inclusive, open sense, meaning that additional elements may be included.

The term “including” is used to mean “including but not limited to”. “Including” and “including but not limited to” are used interchangeably.

The term “elastomer” refers to a polymer that displays rubber-like elasticity.

The term “vulcanization” or “curing” refers to a chemical process for modifying a polymer by forming crosslinks between individual polymer chains.

The acronym “phr” stands for Parts per Hundred Rubber, which is a measure of concentration known in the rubber compounding art. As used herein, “phr” means a proportion of a component per 100 parts of the base elastomer's solid weight.

The term “tensile strength” refers to the maximum amount of tensile stress a material can withstand before breaking.

The term “ultimate elongation” refers to the maximum amount of stretch of a material at break.

The term “modulus” refers to elastic modulus, or the tendency of an object to be deformed elastically when a force is applied to it.

The term “coagulate” refers to a change from a liquid or a sol into a thickened mass. The term “coagulant” refers to an agent that causes a liquid or a sol to coagulate.

The term “MPa” refers to a megapascal, or 1,000,000 Pa. A pascal is a measure of force per unit area. One pascal is equal to one newton per square meter (1 N/m²).

The term “radiation attenuation” refers to the ability to deflect, absorb, etc. the flux of electromagnetic radiation originating from a radiation source and directed towards a patient or medical personnel.

General Description

Provided herein are natural rubber radiation attenuation gloves that meet the higher standards for medical exam and surgical gloves.

Guayule (Parthenium argentatum Gray), a shrub from the American Southwest, produces a circumallergenic natural rubber that is softer and more elastic than traditional hevea natural rubber. The linear guayule natural rubber (GNR) polymer also allows a more integrated polymer filler network than the bulkier branched HNR polymer. This property, in combination with its very low total protein and high fatty acid content, creates more “room” in the matrix enabling higher filler loading. Solid GNR matrix can hold up to three times more bio-based filler than HNR, while still maintaining excellent physical properties.

### EXAMPLES

The experiment shows that the radiation attenuation material filled GNR films meet the tensile requirements of ASTM D3577 or D3578 standards (set forth in Table 1).

Guayule natural rubber is circumallergenic with respect to Type I allergy because its proteins do not cross-react with Hevea-associated allergic proteins induced human antibodies. Guayule is qualified under ASTM D1076-06 Category 4 as a natural rubber latex that contains less than 200 μg protein/g dry weight latex with no detectable protein by ASTM D6499. The extremely low protein content making it very unlikely to induce guayule-specific allergies.

In this example, the guayule natural rubber latex and xanthate based accelerator system was used, as described in Cornish et al. U.S. Ser. No. 14/049,942 filed Oct. 9, 2013 “Rubber Latex Emulsion and Related Methods, Compositions and Articles of Manufacture.” Use of such system allows the films to avoid the skin sensitization rashes (Type IV allergies) and contact dermatitis caused by the common chemical cross-linking accelerators usually used with HNR and synthetic polymers. These added benefits make GNR products ideal for medical uses.

For example, in certain embodiments, the film can be comprised of: guayule natural rubber latex; sulfur; at least one radiation attenuation filler; and, accelerators comprising diisopropyl xanthogen polysulphide (DIXP) and zinc diisononyl dithiocarbamate (ZDNC).

For example, the DIXP can be present at about 2 PHR, and the ZDNC is present at about 0.8 phr. Alternatively, the ZDNC can be present at a dry weight concentration ranging from about 0.01 phr to about 3 phr; and/or, the DIXP can be present at a dry weight concentration ranging from about 0.01 phr to about 5 phr.

In certain embodiments, accelerators comprise diisopropyl xanthogen polysulphide (DIXP) and zinc diisononyl dithiocarbamate (ZDNC), present in a ratio of DIXP:ZDNC of about 2.5:1 or less. In certain embodiments, substantially all of the DIXP is consumed into sulfur crosslinks during a vulcanization process.

In addition, the film can include one or more of: ammonium hydroxide, ZnO, and one or more antioxidants.

In certain embodiments, the film comprises:

about 100 phr rubber of guayule natural rubber;

about 0.01 to about 5 phr of sulfur;

about 120 to about 150 phr of at least one radiation attenuation filler;

about 0.2 to about 1.4 phr of ZDNC; and, about 1 to about 2.2 phr of DIXP.

In certain other embodiments, the film comprises:
about 100 phr of guayule natural rubber;
about 0.01 to about 5 phr of sulfur;

about 120 to about 150 phr of at least one radiation attenuation filler;

about 0.5 to about 1.4 phr of ZDNC; and,

about 1 to about 2.2 phr of DIXP.

In this example, bismuth tri-oxide (Bi₂O₃) is used as an example radiation attenuation filler. Earlier data shows 120 phr loading of Bi₂O₃ at 0.28 mm film thickness provides the minimum radiation attenuation required by ASTM D7866 standard (see Table 2 below). These values were used as a baseline to design the RA medical gloves.

TABLE 2
Specification for Radiation Attenuating Protective Gloves

| | | | | | |
|---|---|---|---|---|---|
| ASTM D7866 | Energy | 60 kVp | 80 kVp | 100 kVp | 120 kVp |
| | Levels | | | | |
| | Minimum | 29% | 22% | 18% | 15% |
| | attenuation | | | | |

Bismuth tri-oxide filler loadings of 120 phr and 150 phr, sulfur loading and vulcanization condition were also varied based on the base GNR compound formulation and tested. Target thickness was set at 28 mm and maintained by compound dipping dwell time. In addition, other factors including former type and the method used to add the Bi₂O₃ filler into compound were analyzed. A list of variables tested and observed is shown in Table 3 below.

TABLE 3
Variables tested and observed

| | | |
|---|---|---|
| Variable | Range | Unit |

| | | | |
|---|---|---|---|
| Sulfur | 0.25 | 0.34 | phr |
| Bi₂O₃ | 120 | 150 | phr |
| Water | 18 | 50 | phr |
| Former | 0.33 | 0.63 | mm |
| Thickness | 0.24 | 0.31 | mm |
| Cure temp | 70 | 105 | ° C. |
| Cure time | 35 | 105 | min |

Formulation—the base compound formulation is shown in Table 4 below.

TABLE 4
Base GNR compounding recipe

| Chemical | phr* |
|---|---|
| GNR latex | 100 |
| NH₄OH | 0.72 |
| Antioxidant | 2.3 |
| ZnO | 0.5 |
| ZDNC | 0.9 |
| DIXP | 1.7 |
| Sulfur | 3.2 |

*phr, parts per hundred rubber dry weight

Compound Preparation

The desired amount of Bi₂O₃ was measured and dispersed by adding various amount of deionized water and mixed thoroughly using a handheld mixer. The compound emulsion without attenuation filler and added water was then prepared by mixing the ingredients. The compound emulsion was then added to the Bi₂O₃ dispersion under slow stirring. Stir speed was gradually increased to make sure Bi₂O₃ was evenly dispersed in the GNR latex compound. The final compound emulsion with Bi₂O₃ was filtered through one layer of 110 mesh silkscreen to remove impurity particles and coagulates. Compound was stored in a 4-10° C. fridge overnight to allow air bubbles to exit, then used within the next 3-5 days until cumulative coagulates of about 10% total Bi₂O₃ weight were removed.

Thin Film Preparation

Thin film samples were produced by dipping coagulant- coated, pre-heated aluminum plate formers into prepared emulsions, followed by heating in a curing oven to remove liquids and vulcanize the GNR. Film thicknesses were controlled by compound dwell time. All thin films were generated with a Diplomat computerized latex dipper.

For each treatment, two film samples were made (identical samples per dip per plate, because both sides of the plate were coated). From each dipping, we chose one side of the film from the plate was chosen for tensile testing, whereas the other side was saved for radiation attenuation testing.

Tensile Measurements

Tensile measurements were performed according to ASTM D412. From the samples chosen, five dumbbell specimens were cut using Die “C”. Specimen thickness was determined as the median of three spots across the testing area measured using a Vernier caliper. The tensile properties of the specimen were determined using a tensiometer (model 3366, Instron, Norwood, Mass., USA) with 50 N static load cell (model 2530-50N, Instron), coupled with a high elongation contact extensometer (model 3800, Epsilon Tech. Corp., Jackson, Wyo., USA). Three key tensile parameters (tensile strength, ultimate elongation, and modulus at 500% strain) were derived from the raw data with the Bluehill program (version 2, Instron).

Former Temperature

Former temperature was measured using a Milwaukee infrared temperature meter. The aluminum plate formers were painted with Rust-oleum spray paint at the non-dipping area, and temperature was measured on the painted area only. Three readings were taken at different spots for each measurement.

Tensile Data Analysis

Analysis of variance was carried out to detect significant variation of key film properties caused by compounding and dipping variables. Inter-treatment comparisons were also conducted to find out statistically significant variations.

Multivariate linear regression was performed to model film tensile performances in response to changes in compounding and processing variables. Factors were manually selected based on ANOVA result and other observations, and were screened by p-value of less than 0.01 for regression modeling.

Several methods were tested to disperse Bi₂O₃ into the GNR latex compound. Adding latex directly into Bi₂O₃ powder caused the GNR latex to coagulate at the latex-Bi₂O₃ interface. This was likely due to latex local dehydration caused by the dense and heavy Bi₂O₃ powder.

Then, the Bi₂O₃ was dispersed in water, then GNR latex or GNR compound was added. This method produced acceptable compound, without large coagulates when filtered through the silk screen.

Amount of added water was altered to determine its effect on dispersing the bismuth tri-oxide (Bi₂O₃) filler. After mixing, 18 phr water and 150 phr Bi₂O₃ was paste-like, and 24 phr water with 150 phr Bi₂O₃ was smoothie-like. As water was increased to 50 phr, the water and Bi₂O₃ phases separated very quickly once stirring stopped. All water loadings resulted acceptable compound.

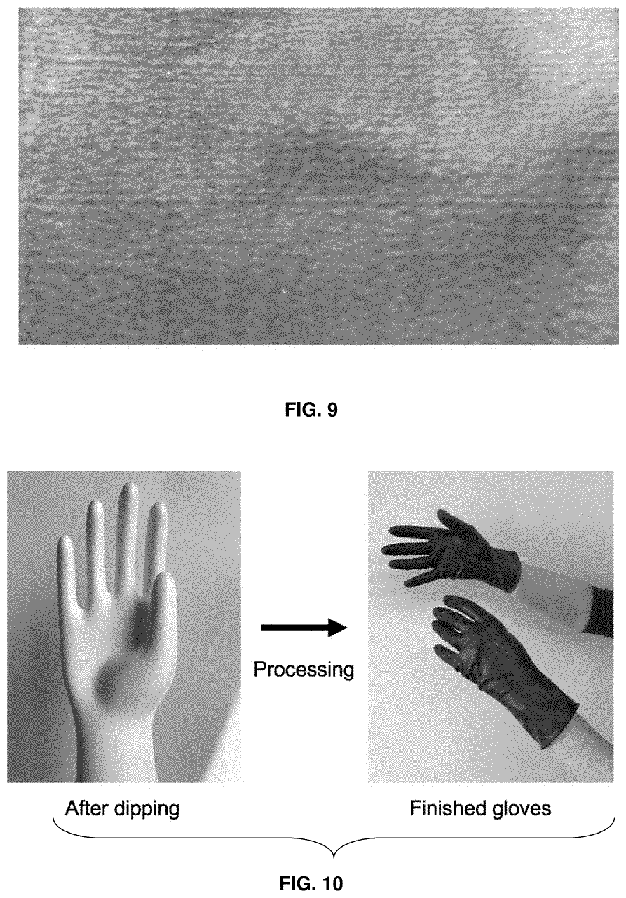

It was also noticed that with 50 phr water, films produced were less vulcanized than lower water content compound under the same vulcanization conditions (by film color, data not shown), likely a result of increased water evaporation time. Water content also affected Bi₂O₃ distribution in resulted films. With 50 phr water, it was observed that many horizontal dotted lines occurred on the non-former-side of films. This was likely the consequence of reduced compound viscosity and increased vulcanization time before the Bi₂O₃ powder particles could be fixed by vulcanized rubber lattices. FIG. 9 showing a film having increased water which caused Bi₂O₃ filler sediment.

Therefore, it is desirable that water be minimized to only moisten the Bi₂O₃, and kept constant to minimize its impact on the vulcanization process.

Film Thickness and Appearance

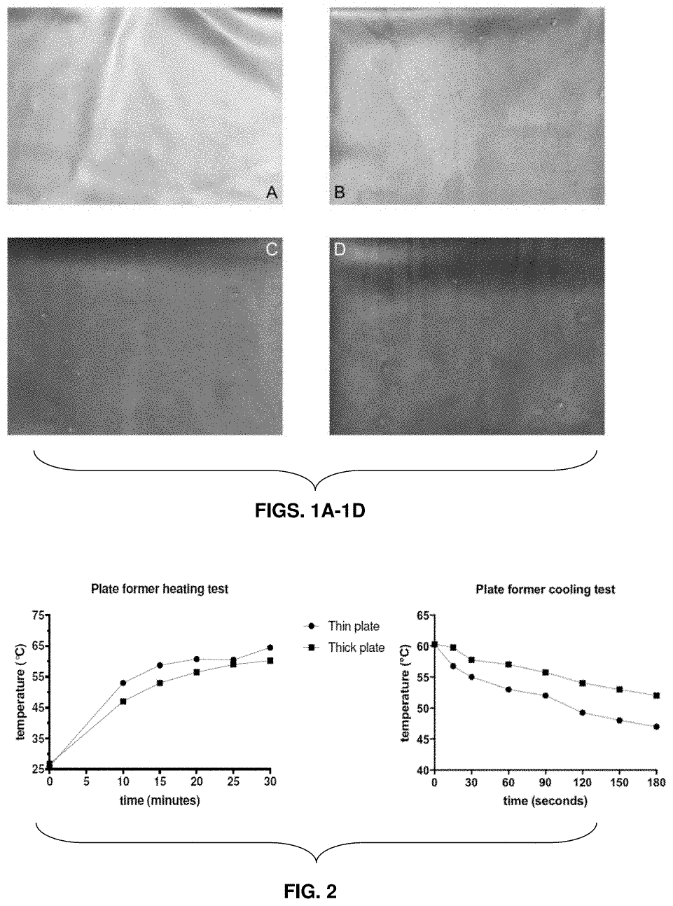

The color of resultant films (former side, same below) ranged from yellow brown to dark brown (FIGS. 1A-1D) and thickness ranged from 0.24 to 0.31 mm (with a mean of 0.285±0.014, n=84).

The color of cured films darkened with increased vulcanization temperature and time (FIG. 1). By manually pulling the films, it was found that the lighter color (FIGS. 1A-1B) films were under-vulcanized as they do not possess proper elasticity (deformed after pulling). Fully vulcanized GNR-Bi₂O₃ films always had a medium to dark brown color on the former side (FIGS. 1C-D). It was used as an indicator for proper vulcanization in later experiments.

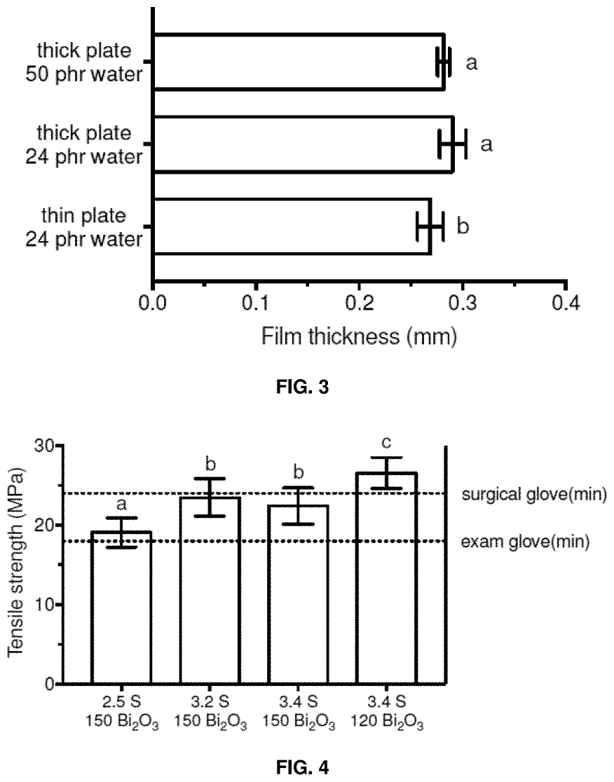

Key factor determining film thickness is dwell time, and 40 s dwell time was used to maintain consistent film thickness of 0.28-0.29 mm. However, it was found that film thickness was also altered by the former used (Error! Reference source not found.FIG.3).

Two types of plate formers were tested first, with thickness of 3.3 mm (thin) and 6.3 mm (thick) respectively. It was observed that thin plate former resulted thinner film (FIG. 3) and higher degree of vulcanization (darker color, data not shown).

The former surface temperature change was measured, and it was found that the thick plate formers heat up and cool down slower than the thin plate formers (FIG. 2). During heating, the thick plate formers had about 5° C. lower surface temperature over the first 30 min monitored. This can be responsible for the different degree of vulcanization of films made on these two former types. During cooling, a difference of 3-5° C. was observed in the first 30-60 seconds and the difference further increased with time. This time frame is after the former is moved out of oven and before it enters compound. During the dwell time, and considering the higher heat capacity of the thick formers, this difference can be further enlarged, affecting amount of compound coagulated onto the former. The film thickness data showed a significant variation of film thicknesses between the two former types (FIG. 3).

This result also indicated that adjustments to the vulcanization process can be made, when transitioning to production with actual ceramic glove formers, due at least in part to the different heat capacity from the aluminum plate formers.

Film Tensile Performance

A total of 84 films were made and tested. The mean and standard deviation for tensile strength was 22.8±3.5 MPa, ultimate elongation was 765.8%±36.5%, and modulus at 500% strain was 3.69±0.82 MPa. Of these 84 samples, 22 passed ASTM D3577 surgical glove standard, and 79 met the ASTM D3578 examination glove standard. Therefore, the further analysis was focused on reproducibly meeting the surgical glove standard for the GNR-Bi₂O₃ RA medical gloves.

Tensile strength is positively correlated with sulfur loading and negatively correlated with bismuth tri-oxide loading (FIG. 4). The 5 samples that failed the exam glove standard were all compounded with 2.5 phr sulfur and 150 phr Bi₂O₃, with tensile strength ranging from 14.4-17.9 MPa. The highest tensile strength obtained was a sample with 3.4 phr sulfur and 120 phr Bi₂O_{3,} with tensile strength of 30.4 MPa.

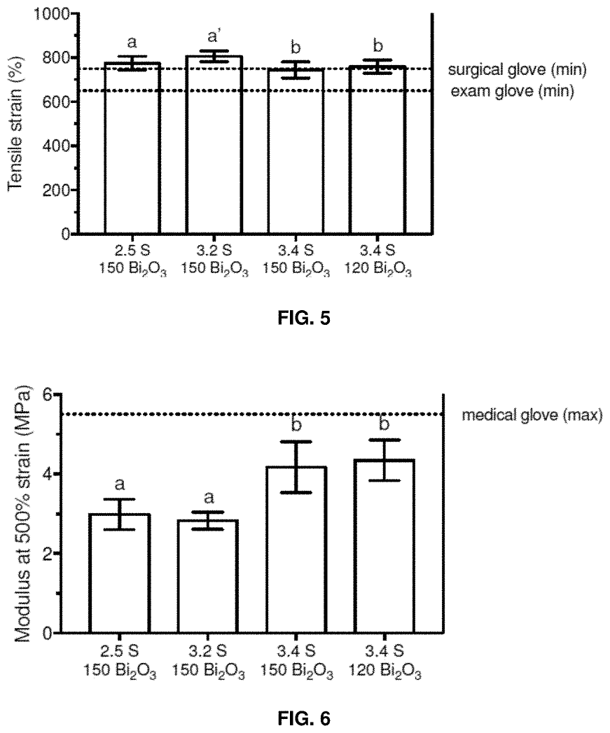

Unexpectedly, instead of the bismuth tri-oxide, only sulfur loading was found to be significantly affected both ultimate elongation and modulus. Ultimate elongation for all samples passed the examination glove standard, over half passed surgical glove standard (FIG. 5). Best ultimate elongation was obtained at 3.2 phr sulfur loading, with 150 phr Bi₂O₃. Elevated sulfur loading also resulted increased modulus (FIG. 6). Though in this example, modulus never exceeded the maximum modulus threshold for both medical glove standards.

These data show that surgical gloves at 120 phr Bi₂O₃ can readily be made with moderate tolerance to vulcanization variations. However, at higher filler loadings (e.g., 150 phr Bi₂O₃), vulcanization conditions play a role, and can be adjusted to accommodate both tensile strength and ultimate elongation.

Vulcanization conditions

Vulcanization temperature and time affect GNR film tensile properties in non-linear pattern. Thus, these two factors were examined by plotting to find the optimal condition that resulted highest tensile properties.

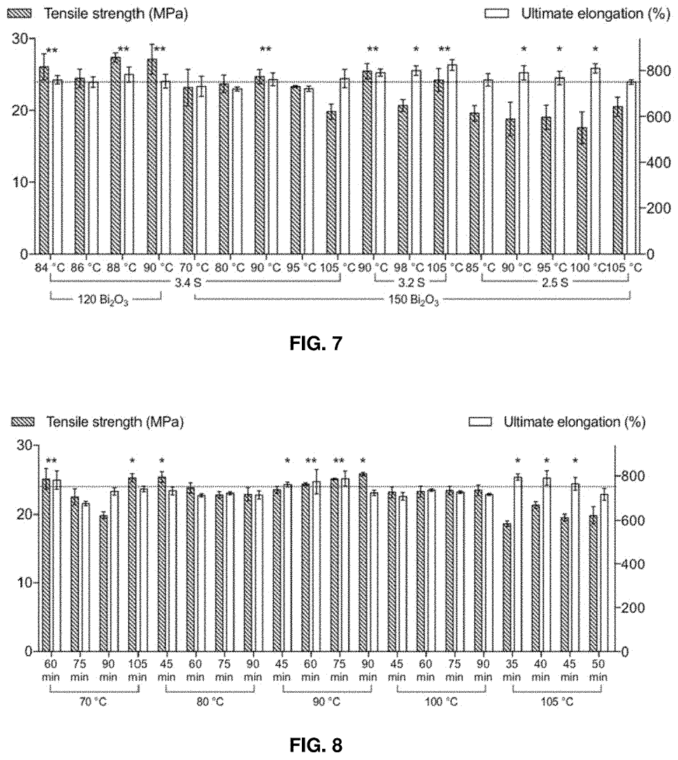

As shown in FIG. 7, the stress and strain data from all samples were analyzed by vulcanization temperature and compounding formulation. Vulcanization temperatures that resulted tensile strength >24 MPa and ultimate elongation >750% were marked with asterisks. In one embodiment, the optimal temperature was determined to be 90° C. as it provided more consistent tensile performances that surpassed the surgical glove standard. Similarly, in another embodiment, the optimal vulcanization time was determined to be 60-75 min at 90° C. (FIG. 8).

Regression Modeling

According to findings from compounding and vulcanization analyses, sulfur loading, bismuth tri-oxide loading, film thickness, vulcanization temperature and time were tested against the three tensile parameters. Because of the non-linearity relationship of vulcanization temperature and time to tensile properties, these two factors were transformed by Ln and multiplied to use as single factor. Derived regression models are listed below.

Tensile strength=15.6+3.74*(S phr)−0.113*(Bi₂O₃phr)+0.638*Ln(vulc. temp)*Ln(vulc. time)

Ultimate elongation=7.46+6.79*(film thickness)−0.096*Ln(vulc. temp)*Ln(vulc. time)

Modulus=−3.3+1.2*(S phr)+0.18*Ln(vulc. temp)*Ln(vulc. time)

Predicted tensile performances were derived based on these models, as shown in Table 5 below.

TABLE 5
Predicted tensile and attenuation performances

| | A | B | C | D | E | F |
|---|---|---|---|---|---|---|
| Sulfur (phr) | 3.2 | 3.3 | 3.4 | 3.2 | 3.4 | 3.6 |
| Bi₂O₃ (phr) | 120 | 140 | 140 | 150 | 180 | 200 |
| Cure temp (° C.) | 90 | 90 | 90 | 90 | 90 | 90 |
| Cure time (min) | 70 | 70 | 70 | 70 | 70 | 70 |
| Thickness (mm) | 0.28 | 0.28 | 0.3 | 0.28 | 0.3 | 0.3 |
| Tensile strength | 26.20 | 24.32 | 24.69 | 22.81 | 20.17 | 18.66 |
| (MPa) | | | | | | |
| Ultimate | 752% | 752% | 766% | 756% | 766% | 766% |
| elongation | | | | | | |
| Modulus at 500% | 3.98 | 4.10 | 4.22 | 3.98 | 4.22 | 4.46 |
| (MPa) | | | | | | |
| Estimated | 100% | 117% | 125% | 127% | 161% | 179% |
| attenuation | | | | | | |
| (% to ASTM | | | | | | |
| baseline) | | | | | | |

Radiation attenuation from baseline attenuation levels required by ASTM D7866 was also estimated based on Bi₂O₃ loading proportional to 120 phr and film thickness proportional to 28 mm.

It is demonstrated herein that prototype guayule natural rubber radiation attenuation gloves meet medical examination and surgical glove standards.

The unique physical properties of GNR latex provide an improved radiation attenuation glove. This is especially important since the current limited GNR latex production capacity does not permit large quantities to be supplied to commodity manufacturers.

It is also demonstrated herein that the distinct tensile profile of GNR latex coupled with its high filler capacity, provides additional innovative products, and provides considerable value to the advancement of existing dipped rubber product industries.

Variable Parameters

Vulcanization condition is altered by former heat capacity and likely oven heating speed and capacity. Consequently, in a production setting, various parameters can be adjusted for further optimization to reproducibly achieve surgical glove performance of guayule RA gloves with higher Bi₂O₃loading. Water content of compound also affects vulcanization condition, something to be noted as Bi₂O₃ dispersion method may also vary during scale up.

Variable Fillers

In other embodiments, the RA gloves can contain different fillers, such as micro- to nano-grade powder forms of bismuth tri-oxide; Bi₂O₃), barium sulfate (BaSO₄), barium carbonate (BaCO₃), tungsten tri-oxide (WO₃), and tungsten (W).

Also, in other embodiments, the RA gloves can contain filler combinations where there is a mix certain ratios of fillers to achieve optimal attenuation, capitalizing on respective peak radiation distinction characteristics. The benefit of such different fillers and/or combinations as compared to a single filler compound is the reduction in total filler loading for similar attenuation levels, thus achieving better tensile performance, and a reduction in filler cost.

Other Ingredients

The guayule natural rubber films of the present disclosure are cured with the accelerators diisopropyl xanthogen polysulphide (DIXP) and zinc diisononyl dithiocarbamate (ZDNC). DIXP is consumed during the vulcanization process, and skin tests have shown that ZDNC does not cause dermal reactions or delayed contact hypersensitivity, thus eliminating Type IV allergy sensitization.

In addition, DIXP contains no nitrogen, phosphorus, or metallic elements, making it unable to form the volatile and carcinogenic N-nitrosamine compounds during vulcanization. This further reduces occupational hazards for latex industry workers and product users.

Accelerators and activators are generally used in vulcanization processes to lower the activation energy of the vulcanization reaction. ZDNC and DIXP are alternative accelerators that utilize sulfur but do not leave residual chemicals associated with Type IV allergy. ZDNC has a lower allergenic potential than conventional dithiocarbamates because its high molecular weight renders it soluble in the rubber matrix. ZDNC does not bloom to the surface of latex films, and therefore less ZDNC can be extracted from finished rubber articles compared to common industry accelerators such as zinc dibenzyldithiocarbamate (ZBEC). DIXP, a fugitive xanthate accelerator, is advantageous over other fugitive accelerators because it is consumed completely into sulfur crosslinks, and its byproducts are volatile isopropanol and carbon disulfide. DIXP contains no nitrogen, and therefore cannot form the volatile and carcinogenic N-nitrosamines associated with thiuram and dithiocarbamate accelerators. However, DIXP cannot, as a sole accelerator, sufficiently accelerate sulfur crosslinks to generate good tensile properties. Therefore, the present disclosure utilizes DIXP in conjunction with ZDNC as the accelerators with GNR latex to create truly circumallergenic natural rubber thin films. The resulting circumallergenic thin films have protein levels generally ranging from about 0 to about 2 μg extractable protein/g latex film.

The sulfur can be elemental sulfur or sulfur-containing compounds. Suitable sulfur components include, but are not limited to: sulfur powder; precipitated sulfur; colloidal sulfur; insoluble sulfur; high-dispersible sulfur; sulfur halides such as sulfur monochloride and sulfur dichloride; sulfur donors such as 4,4′- dithiodimorpholine; sulfur dispersions; amine disulfides; polymeric polysulfides; aromatic thiazoles; amine salts of mercaptobenzothiazoles; and combinations thereof. In particular embodiments, the sulfur is a sulfur dispersion. By way of non-limiting example, sulfur dispersions can be prepared by mixing elemental sulfur with a resin and a solvent.

In certain embodiments, the dry weight concentration of the GNR latex ranges from about 1 phr to about 100 phr. In particular embodiments, the GNR latex is present at a concentration of about 100 phr. In certain embodiments, the dry weight concentration of the sulfur ranges from about 0.01 phr to about 5 phr, from about 0.1 phr to about 3.5 phr, or from about 1 phr to about 3 phr. In particular embodiments, the sulfur is present at a concentration of about 2 phr.

In certain embodiments, the dry weight concentration of the ZDNC ranges from about 0.01 phr to about 3 phr, from about 0.1 phr to about 2 phr, or from about 0.2 phr to about 1.4 phr. In particular embodiments, the ZDNC is present at a concentration of 0.2 phr, 0.4 phr, 0.6 phr, 0.8 phr, 1.0 phr, 1.2 phr, or 1.4 phr. In certain embodiments, the dry weight concentration of the DIXP ranges from about 0.01 phr to about 5 phr, from about 0.1 phr to about 3 phr, or from about 1 phr to about 2.2 phr. In particular embodiments, the DIXP is present at a concentration of 1 phr, 1.2 phr, 1.4 phr, 1.6 phr, 1.8 phr, 2.0 phr, 2.1 phr, or 2.2 phr.

In certain embodiments, the GNR latex compound further comprises one or more of ammonium hydroxide, ZnO, and an antioxidant. All ingredients may be in the form of a dispersion. Suitable antioxidants include any phenolic antioxidant capable for use in latex manufacturing. When present, the dry weight concentration of the ammonium hydroxide ranges from about 0.01 phr to about 5 phr, from about 0.1 phr to about 3 phr, or from about 0.8 phr to about 2 phr. In particular embodiments, ammonium hydroxide is present at a concentration of about 1 phr. When present, the dry weight concentration of the ZnO ranges from about 0.01 phr to about 2 phr, from about 0.1 phr to about 1 phr, or from about 0.2 phr to about 0.5 phr. In particular embodiments, ZnO is present at a concentration of about 0.3 phr. When present, the dry weight concentration of the antioxidants ranges from about 0.01 phr to about 5 phr, from about 0.1 phr to about 4 phr, or from about 1 phr to about 3 phr. In particular embodiments, the antioxidants are present at a concentration of about 2 phr.

Examples of Articles

The natural rubber latex thin films described herein circumvent Type I and/or Type IV latex allergies, are nitrosamine free, and have outstanding performance characteristics. Therefore, the thin films are useful in a wide variety of fabricated articles. By way of non-limiting example, the circumallergenic or hypoallergenic natural rubber thin films of the present disclosure can be fabricated into, or otherwise applied in the fabrication of: surgical gloves, examination gloves, personal protective gloves, radiation shielding thyroid shield, radiation shielding apron, and other radiation protective or shielding garments. Many other applications of the radiation attenuation thin films are envisioned and within the scope of the present disclosure.

The articles described herein can be fabricated from the thin films in any of a variety of fabrication methods. These methods include, but are certainly not limited to, coagulant dipping processes, straight-dipping processes, casting processes, and foaming processes. For example, FIG. 10 shows the appearance of example GNR-Bi₂O₃ RA medical gloves.

Though dipping processes have been specified to exemplify certain aspects of the articles made from the thin films, the skilled practitioner will understand that casting and foaming processes for making circumallergenic films and articles are entirely within the scope of the present disclosure.

Example of Methods of Making

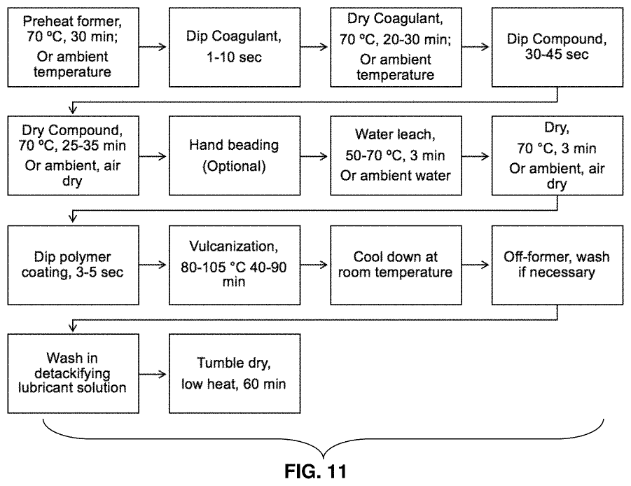

FIG. 11 shows one example of a factory scale GNR-RA glove fabrication process. The method can include the following steps:
1a) preheat former at about 70° C. for about 30 minutes, or
1b) start process at ambient temperature;
2) dip former into coagulant (one example is a water based calcium nitrate solution to coat the glove former and coagulate the latex, consisting of 20%-40% Calcium nitrate, 0.5%-2% Zinc stearate, 0.5%-2% Triton X100). Others can be used, such as 20% calcium nitrate in methanol, or 20% acetic or formic acid in methanol) for about 1 to about 10 seconds;
3a) dry coagulant on former at about 70° C. for about 20 to about 30 minutes, or
3b) dry coagulant on former at ambient temperature;
4) dip former with dried coagulant into compound (the compounds included those already described, herein). In other embodiments, it is possible to make this glove with the conventional accelerators as well, but that would leave the glove with contact sensitizers) for about 30 to about 45 seconds;
5a) dry compound on the former at about 70° C. for about 25 to about 35 minutes, or
5b) dry compound on the former at ambient temperature;
6) optionally, perform hand beading;
7a) water leach at about 50 to about 70° C. for about 3 minutes, or
7b) water leach at ambient temperature for about 3 minutes;
8a) dry at about 70° C. for about 3 minutes, or
8b) dry at ambient temperature for about 3 minutes;
9) dip into polymer coating (one example is a water based polyurethane dispersion to improve donning, consisting of 0.5%-5% dry weight polyurethane, 0.5-5% dry weight polychloroprene, less than 0.5% polyurethane crosslinker, less than 0.5% antioxidant. Donning agents are used to replace powder, which has recently been banned by FDA, such that the glove can still be easily put onto to hand. More examples of suitable materials include polyurethane with or without silicone, nitrile, methacrylate, acrylate terpolymer. As an alternative to polymer coating, the glove can be chlorinated) for about 3 to about 5 seconds;
10) vulcanize at about 80 to about 100° C. for about 40 to about 90 minutes;
11) cool down to ambient temperature;
12) remove glove from former, wash if necessary;
13) wash glove in detackifying lubricant solution (one example is a water based dimethicone dispersion used to reduce glove overall tackiness and improve donning, consisting of 0.5%-5% Dimethicone emulsion. Many others are available, including anionic paraffin/polyethylene wax emulsion, anionic carnuba wax emulsion, coemulsions of paraffin and wax; and,
14) tumble dry glove at low heat for about 60 minutes.

Certain embodiments of the thin films and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A thin film, comprising: guayule natural rubber, sulfur, and a radiation attenuation filler,
the film having a thickness of about 0.08 to about 0.40 mm,
the thin film having a percent attenuation of at least about 29% at 60 kVp, at least about 22% at 80 kVp, at least about 18% at 100 kVp, and at least about 15% at 120 kVp; and
the film being formed at curing temperatures of about 80 to about 105° C. for about 40 to about 90 minutes.

2. The film of claim 1, wherein the sulfur is present at about 3.2 to about 3.6 per hundred rubber (phr).

3. The film of claim 1, wherein the radiation attenuation filler is present at about 120 to about 200 phr.

4. The film of claim 1, wherein the attenuation filler comprises one or more of: bismuth tri-oxide (Bi₂O₃), barium sulfate (BaSO₄), barium carbonate (BaCO₃), tungsten tri-oxide (WO₃), and tungsten (W).

5. The film of claim 1, wherein the attenuation filler comprises Bi₂O₃ at about 120 to about 200 phr.

6. The film of claim 1, wherein the film has a thickness of about 0.24 mm to about 0.31 mm.

7. A radiation attenuation examination glove comprising the film of claim 1, wherein the film has:
a thickness of about 0.08 to about 0.40 mm,
a tensile strength of at least about 18 MPa,
an elongation at break of at least about 650%, and
a 500% modulus of at most 5.5 MPa.

8. A radiation attenuation surgical glove comprising the film of claim 1, wherein the film has:
a thickness of about 0.10 to about 0.40 mm,
a tensile strength of at least about 24 MPa,
an elongation at break of at least about 750%, and
a 500% modulus of 5.5 at most MPa.

9. The film of claim 1, further including accelerators comprising diisopropyl xanthogen polysulphide (DIXP) and zinc diisononyl dithiocarbamate (ZDNC).

10. The film of claim 11, wherein the DIXP is present at about 2 PHR, and the ZDNC is present at about 0.8 phr.

11. The film of claim 11, wherein the ZDNC can be present at a dry weight concentration ranging from about 0.01 phr to about 3 phr.

12. The film of claim 11, wherein the DIXP is present at a dry weight concentration ranging from about 0.01 phr to about 5 phr.

13. The film of claim 11, wherein DIXP and ZDNC are present in a ratio of DIXP:ZDNC of about 2.5:1 or less.

14. The film of claim 11, further including one or more of: ammonium hydroxide, ZnO, and one or more antioxidants.

15. The film of claim 11, comprising:
about 100 phr rubber of guayule natural rubber;
about 0.01 to about 5 phr of sulfur;
about 120 to about 150 phr of at least one radiation attenuation filler;
about 0.2 to about 1.4 phr of ZDNC; and
about 1 to about 2.2 phr of DIXP.

16. The film of claim 11, comprising:
about 100 phr of guayule natural rubber;
about 0.01 to about 5 phr of sulfur;
about 120 to about 150 phr of at least one radiation attenuation filler;
about 0.5 to about 1.4 phr of ZDNC; and
about 1 to about 2.2 phr of DIXP.

17. A method for making a glove, comprising:
combining guayule natural rubber latex, sulfur, and an attenuation filler, and
forming the glove by dipping followed by curing at about 80 to about 105° C. for about 40 to about 90 minutes.

18. A method for making a glove, comprising the following steps:
1) preheat former at about 70° C. for about 30 minutes,
2) dip former into coagulant for about 1 to about 10 seconds;
3) dry coagulant on former at about 70° C. for about 20 to about 30 minutes;
4) dip former with dried coagulant into compound for about 30 to about 45 seconds;
5) dry compound on the former at about 70° C. for about 25 to about 35 minutes;
6) optionally, perform hand beading;
7) water leach at about 50 to about 70° C. for about 3 minutes,
8) dry at about 70° C. for about 3 minutes;
9) dip into polymer coating for about 3 to about 5 seconds;
10) vulcanize at about 80 to about 100° C. for about 40 to about 90 minutes;
11) cool down to ambient temperature;
12) remove glove from former, wash if necessary;
13) wash glove in detackifying lubricant solution; and,
14) tumble dry glove at low heat for about 60 minutes.

19. A method for making a glove, comprising the following steps:
1 a) preheat former at about 70° C. for about 30 minutes, or
1b) start process at ambient temperature;
2) dip former into coagulant for about 1 to about 10 seconds;
3a) dry coagulant on former at about 70° C. for about 20 to about 30 minutes, or
3b) dry coagulant on former at ambient temperature;
4) dip former with dried coagulant into compound for about 30 to about 45 seconds;
5a) dry compound on the former at about 70° C. for about 25 to about 35 minutes, or
5b) dry compound on the former at ambient temperature;
6) optionally, perform hand beading;
7a) water leach at about 50 to about 70° C. for about 3 minutes, or
7b) water leach at ambient temperature for about 3 minutes;
8a) dry at about 70° C. for about 3 minutes, or
8b) dry at ambient temperature for about 3 minutes;
9) dip into polymer coating for about 3 to about 5 seconds;
10) vulcanize at about 80 to about 100° C. for about 40 to about 90 minutes;
11) cool down to ambient temperature;
12) remove glove from former, wash if necessary;
13) wash glove in detackifying lubricant solution; and,
14) tumble dry glove at low heat for about 60 minutes.
